(19) United States
(12) Patent Application Publication
(10) Pub. No.: US 2025/0166726 A1
(43) Pub. Date: May 22, 2025

(54) STRATICATION USING MULTI-MODAL PREDICTIVE FEATURES

(71) Applicant: International Business Machines Corporation, Armonk, NY (US)

(72) Inventors: Pablo Meyer Rojas, Brooklyn, NY (US); Daniel Enoch Platt, Putnam Valley, NY (US); Akira Koseki, Yokohama-shi (JP); Toshiya Iwamori, Tokyo (JP); Sanjoy Dey, White Plains, NY (US); Michiharu Kudo, Kamakura-shi (JP); Simona Rabinovici-Cohen, Haifa (IL); Itai Guez, Karmiel (IL)

(21) Appl. No.: 18/514,303

(22) Filed: Nov. 20, 2023

(51) Int. Cl. G16B 20/20; G16B 40/00

(52) U.S. Cl. CPC G16B 20/20; G16B 40/00

(57) ABSTRACT

Stratification using multi-modal predictive features constructs unimodal models, where a unimodal model is trained based on a mode of data that is different from another mode of data used to train another unimodal model. The unimodal models are trained to extract features that are predictive of a health condition. The unimodal models are run that extract sets of features, where each set of the sets of features are associated with a mode of data. For each set of the sets of features, the features in the set are ranked, stratification of a population according to a top ranked feature defines a subpopulation, genome-wide association study (GWAS) using genomic data associated with the subpopulation is performed, and at least one genomic variant associated with the health condition is identified based on the performed GWAS.

### BACKGROUND

The present application relates generally to computers and computer applications, and more particularly to machine learning, genome-wide association study, and population stratification using multi-modal predictive features.

### BRIEF SUMMARY

The summary of the disclosure is given to aid understanding of a computer system and method of stratification using multi-modal predictive features, and not with an intent to limit the disclosure or the invention. It should be understood that various aspects and features of the disclosure may advantageously be used separately in some instances, or in combination with other aspects and features of the disclosure in other instances. Accordingly, variations and modifications may be made to the computer system and/or their method of operation to achieve different effects.

A computer-implemented method, in some embodiments, includes constructing a plurality of unimodal models, a unimodal model of the plurality of unimodal models trained based on a mode of data that is different from another mode of data used to train another one of the plurality of unimodal models, the plurality of unimodal models being trained to extract features that are predictive of a health condition. The computer-implemented method also includes running the plurality of unimodal models, where sets of features are extracted, each set of the sets of features being associated with a mode of data. The computer-implemented method also includes, for each set of the sets of features, ranking the features in the set, performing a stratification of a population into a subpopulation according to a top ranked feature, where the top ranked feature is used as a special trait that has predictive association with the health condition, performing a genome-wide association study (GWAS) using genomic data associated with the subpopulation, and identifying, based on the performed GWAS, at least one genomic variant associated with the health condition.

A system, in some embodiments, includes at least one memory device. The system also includes at least one computer processor. The at least one computer processor is configured to construct a plurality of unimodal models, a unimodal model of the plurality of unimodal models trained based on a mode of data that is different from another mode of data used to train another one of the plurality of unimodal models, the plurality of unimodal models being trained to extract features that are predictive of a health condition. The at least one computer processor is also configured to run the plurality of unimodal models, where sets of features are extracted, each set of the sets of features being associated with a mode of data. The at least one computer processor is also configured to, for each set of the sets of features, rank the features in the set, perform a stratification of a population into a subpopulation according to a top ranked feature, where the top ranked feature is used as a special trait that has predictive association with the health condition, perform a genome-wide association study (GWAS) using genomic data associated with the subpopulation, identify, based on the performed GWAS, at least one genomic variant associated with the health condition.

A computer readable storage medium storing a program of instructions executable by a machine to perform one or more methods described herein also may be provided.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1 shows an example of a computing environment, which can implement stratification of populations using multi-modal predictive features in some embodiments.

FIG. 2 is a flow diagram illustrating a method of stratification using multi-modal predictive features in some embodiments.

FIGS. 3A-3B illustrate population stratification using multimodal features in some embodiments.

FIGS. 4A-4B illustrate an overview of GWAS on stratified population in some embodiments.

FIGS. 5A-5B illustrate a case study of an example population in some embodiments.

FIG. 6 is a diagram showing components of a system that can implement stratification of population using features of multi-modal data in some embodiments.

### DETAILED DESCRIPTION

A computer-implemented method, in some embodiments, includes constructing a plurality of unimodal models, a unimodal model of the plurality of unimodal models trained based on a mode of data that is different from another mode of data used to train another one of the plurality of unimodal models, the plurality of unimodal models being trained to extract features that are predictive of a health condition. The computer-implemented method also includes running the plurality of unimodal models, where sets of features are extracted, each set of the sets of features being associated with a mode of data. The computer-implemented method also includes, for each set of the sets of features, ranking the features in the set, performing a stratification of a population into a subpopulation according to a top ranked feature, where the top ranked feature is used as a special trait that has predictive association with the health condition, performing a genome-wide association study (GWAS) using genomic data associated with the subpopulation, and identifying, based on the performed GWAS, at least one genomic variant associated with the health condition. In this way, for example, new and more reliable SNP variants may be discovered.

One or more of the following features can be separable or optional from each other.

In some embodiments, the computer-implemented method further includes retraining each of the plurality of unimodal models based on the respective subpopulation defined during the stratification. The computer-implemented method further includes repeating the running, the ranking, the performing of the stratification, the performing of the GWAS, and the identifying steps. In this way, for example, more targeted features from a less heterogeneous or more focused population related to the health condition may be extracted.

In some embodiments, the computer-implemented method further includes iteratively performing the retraining and the repeating until the subpopulation meets a threshold population size. In this way, for example, more homogenous population related to the health condition can be identified for study.

In some embodiments, the performing of the stratification, the performing of the GWAS, and the identifying steps are performed for a threshold number of next top ranked features as the top ranked feature. In this way, for example, targeted subpopulations based on different top features can be identified.

In some embodiments, the computer-implemented method further includes performing gene ontology enrichment analysis to discover biological functions associated to single nucleotide polymorphism (SNPs) using data associated with the subpopulation. For example, biological functions can be discovered, which are associated to significant SNPs by using data associated with the subpopulation and performing gene ontology enrichment analysis.

In some embodiments, multiple modes of data, each of which is used to train a respective different unimodal model in the plurality of unimodal models, include at least image data and clinical data. In this way, for example, traits (e.g., phenotypes) can be extracted from predictive features, e.g., those predictive of the health condition. For example, phenotype heterogeneity can be addressed by using multimodal data that includes at least image data and clinical data to perform stratification of a target population.

In some embodiments, the health condition is a disease. In this way, for example, traits related to various disease can be identified, thereby providing more ways to possibly prevent those disease, e.g., allowing preventive mechanisms to be developed.

In some embodiments, the plurality of unimodal models are combined as a multimodal model, where the running the plurality of unimodal models includes running the multimodal model. For example, in this way multiple unimodal models can be run together as a multimodal model.

A system including at least one computer processor and at least one memory device coupled with the at least one computer processor is also disclosed, where the at least one computer processor is configured to perform one or more methods described above. A computer program product is also disclosed that includes a computer readable storage medium having program instructions embodied therewith, where the program instructions are readable by a device to cause the device to perform one or more methods described above.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as stratification using multi-modal predictive features algorithm code 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IOT) sensor set 125), and network module 115. Remote server 104 includes remote database 130. Public cloud 105 includes gateway 140, cloud orchestration module 141, host physical machine set 142, virtual machine set 143, and container set 144.

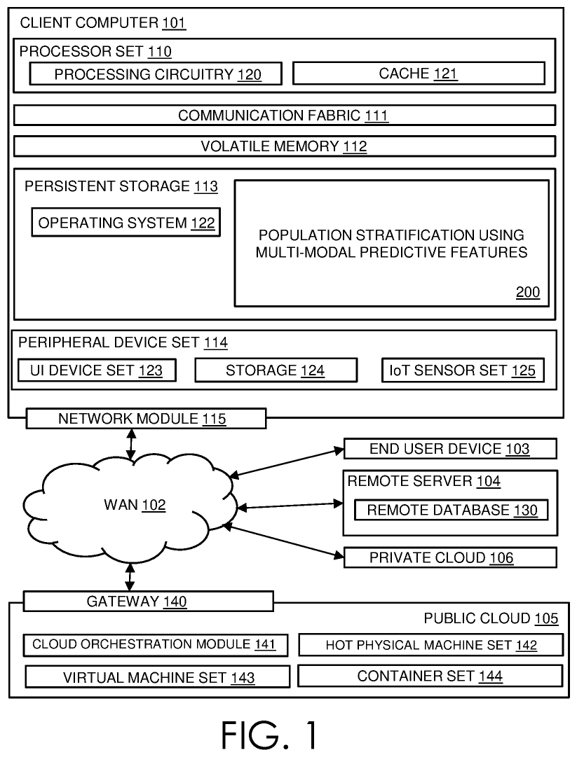

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

A setup for genome-wide association study (GWAS), for example, Single Nucleotide Polymorphism (SNP) enrichment analysis, includes grouping subjects according to phenotypes or traits (e.g., observable characteristics of an individual). Non-genetic variables such as clinical data, laboratory measurements including levels of metabolites and chemical entities or imaging of body variables can also be considered as phenotypes and can be used for GWAS. GWAS and SNP enrichment analysis can become complex, in part due to occurrence of rare variants, complex relationships among SNPs (epistatic effect), and heterogeneity of the phenotype.

In some embodiments, one or more systems and/or methods extract predictive features, and use those predictive features as special phenotypes or traits for grouping subjects, that is, stratification of a population for GWAS. For example, in some embodiments, a system and/or method address phenotypic heterogeneity by using a multimodal data approach to stratify a target population using one or more of the most predictive non-genetic predictive variables from multimodal data features of a model predicting an outcome of interest. Based on such stratification, the system and/or method may then discover new and more reliable SNP variants.

In some embodiments, a system and/or method can link medications and clinical conditions in a more accurate manner. For example, the system and/or method may help in discoveries such as links between certain medications (or combinations of medications) and physiological disorders.

In some embodiments, a system and/or method performs groupings or stratifications of patient populations by selecting features output from one or more predictive models, using the features to group subjects, where features can be determined from multimodal data, and using the groups to discover new associated SNPs.

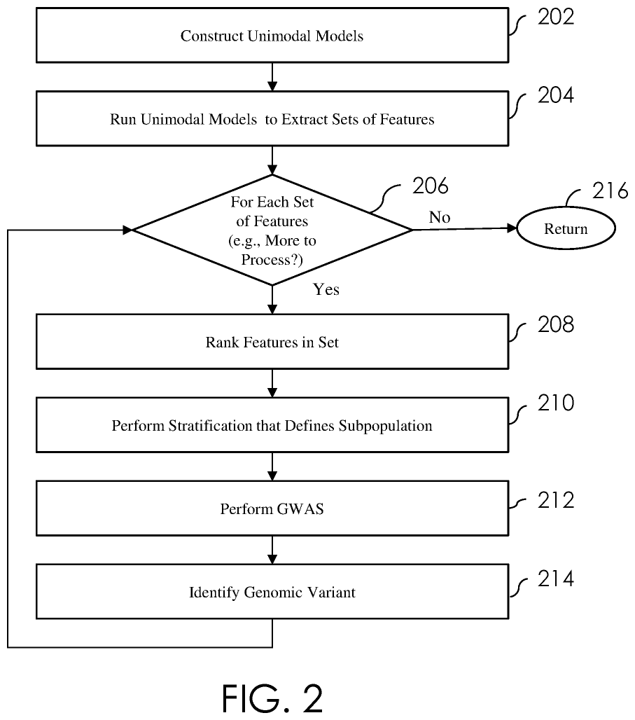

FIG. 2 is a flow diagram illustrating a method of stratification using multi-modal predictive features in some embodiments. One or more computer processors, for example, but not limited to, described above with reference to FIG. 1, can implement the method. At 202, the method includes constructing a plurality of unimodal models. A unimodal model of the plurality of unimodal models is trained based on a mode of data that is different from another mode of data used to train another one of the plurality of unimodal models. In some embodiments, multiple modes of data, each of which is used to train a respective different unimodal model in the plurality of unimodal models, include at least image data and clinical data. For example, several unimodal models are constructed. Each unimodal model can be constructed using a different data type. A data type is also referred to as a mode of data. The plurality of unimodal models are trained to extract features that are predictive of a health condition. In some embodiments, the health condition is a disease.

At 204, the method includes running the plurality of unimodal models, where sets of features are extracted, each set of the sets of features being associated with a mode of data. For example, running a unimodal model using input data of a data type (mode of data) generates or outputs a set of features associated with that data type (mode of data). For instance, a unimodal model that receives as input an image data, outputs or extracts a set of features from that image data; Another unimodal model that receives as input a clinical data, outputs or extracts a set of features from that clinical data.

In some embodiments, multi-modals can be constructed by using each of the different data types to construct a model and then integrating overall model predictions, for example, using a model fusion approach. For example, in some embodiments, the plurality of unimodal models are combined as a multimodal model. From the multimodal model, an output of a ranked sets of features by their importance can be obtained. In this example, running the plurality of unimodal models includes running the multimodal model post-hoc for late fusion or using unimodal features for early fusion.

Processing at 208, 210, 212 and 214 are performed for each set of features in the sets of features extracted from running the plurality of unimodal models at 204. For example, at 206, if there are sets of features to process, the method proceeds to 208. Otherwise, the method returns at 216.

At 208, the method includes ranking the features in the set. Features can be ranked according to their importance as related to how well each feature predicts the health condition. For example, in some cases patient's demographic attribute can be an important feature of the health outcome, but other patient's data such as heart condition and blood pressure can be more important features than the demographic attribute, that is, having their value allows a better prediction of the patient health condition/trait outcome than having only the demographic attribute.

At 210, the method includes performing a stratification of a population into subpopulation according to the top ranked feature. For example, subjects in the population having a certain top ranked feature is grouped into a subpopulation (e.g., all patients older than Y years, with a heart condition, and/or with high blood pressure). Thus, for example, a top ranked feature is used as a special trait that has predictive association with the health condition and used to define a subpopulation. For instance, a subject having that special trait can be considered to have a higher probability of having the health condition. In some embodiments, a top feature can be an attribute. In another embodiment, a top feature can be a combination of attributes.

At 212, the method includes performing a genome-wide association study (GWAS) using genomic data associated with the subpopulation.

At 214, the method includes identifying, based on the performed GWAS, at least one genomic variant associated with the health condition.

In some embodiments, the method also includes retraining each of the plurality of unimodal models based on the respective subpopulation defined during the stratification. For example, the data associated with the subpopulation can be used to retrain a unimodal model, where the unimodal model learns to extract features from that data. Since a different set of training data (e.g., the subpopulation's data) are used to retrain the unimodal model, the unimodal model may extract or rank a set of features differently, e.g., establishing different relationships between features and the health condition. Running of the unimodal model (which is retrained) 204, ranking of the features 208, and performing of the stratification 210 can be repeated. Performing GWAS 212 and identifying at least one genomic variant 214 can also be repeated. In this way, for example, more targeted features coming from a less heterogeneous or more focused population related to the health condition may be extracted.

In some embodiments, the method also includes iteratively performing retraining of the unimodal models based on respective subpopulations, running of the unimodal models (e.g., similarly as done at 204, using retrained unimodal models), and for each set of features obtained at 204, ranking of the features (e.g., similarly as done at 208), and performing of the stratification (e.g., similarly as done at 210), until the subpopulation meets a threshold population size. In this way, for example, more homogenous population related to the health condition can be identified for study.

In some embodiments, performing of the stratification (e.g., similarly as done at 210), performing of the GWAS (e.g., similarly as done at 212), and identifying of at least one genomic variant (e.g., similarly as done at 214) are performed for a threshold number of next top ranked features as the top ranked feature. For instance, those processing can be performed for each of a number of top ranked features. The threshold number can be predefined or preconfigured, for example, by a subject matter expert.

In some embodiments, the method includes performing a gene ontology enrichment analysis to discover associated genetic mechanisms from the enriched single nucleotide polymorphism (SNPs) using data associated with the subpopulation.

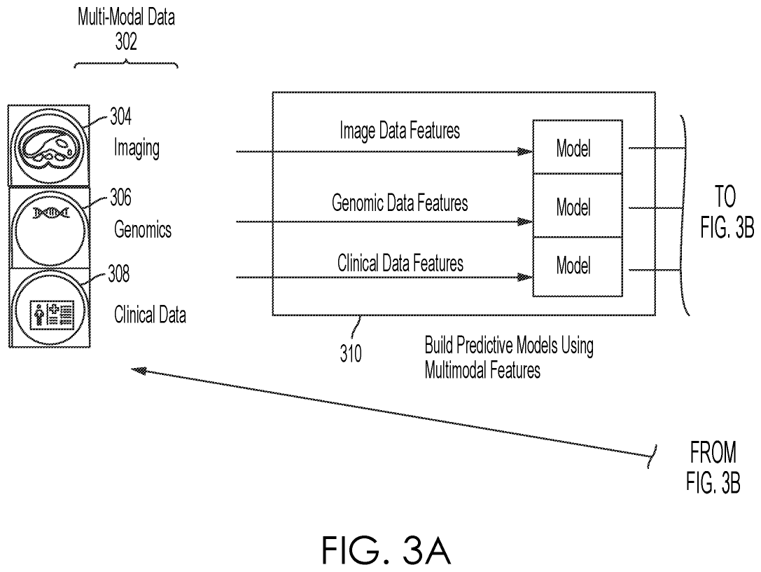

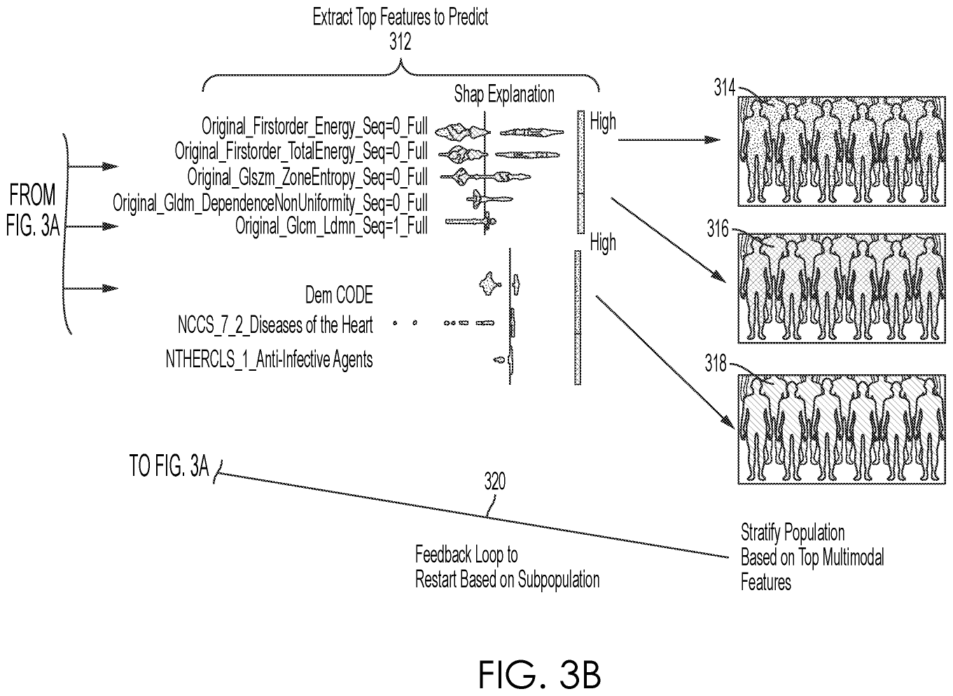

FIGS. 3A-3B show a diagram illustrating population stratification using multimodal features in some embodiments. One or more computer processors can perform operations or functions shown in the figure. For example, a processor uses multi-modal data 302 such as imaging data 304, genomics data 306, and clinical data 308 to build or construct predictive models 310. Other modes of data can be employed such as demographics or wearable data. Therefore, the methodology disclosed herein is not limited to the three types or modes of data shown in the figure.

For example, several unimodal models are constructed. Each unimodal model is constructed using data from a mode of data. For example, using features from image data 304 such as magnetic resonance imaging (MRI) data, a unimodal model (e.g., referred to as a second unimodal model for explanation only) is constructed and trained to extract image features that are predictive of a health condition. For example, a supervised learning can be performed where the unimodal model is given a training dataset that includes images of subjects that have the health condition and images of subjects that do not have the health condition. Based on such labeled image data set, the unimodal model is trained to predict and hence learns to extract important predictive features from the images related to the health condition, for example, features that the subjects with the health condition have in common, but not present in the images of the subjects without the health condition. Those features are then considered predictive of the health condition. For instance, subjects having disease A can have an enlarged component H present in their brain, whole body, and/or organ images, compared to subjects who do not have disease A. Enlarged component H then can be extracted as a feature that is predictive of disease A. An example of a unimodal model includes, but is not limited to, a deep learning model with convolution neural networks (CNN) that can process image data.

Similarly, using genomic data, a unimodal model is constructed and trained to extract genomic features that are predictive of the heath condition. For example, a training dataset that includes SNPs derived from the genomic data of subjects that have the health condition and those that do not have the health condition can be used to train the unimodal model to extract genomic features that are predictive of the health condition. Examples of a unimodal model include but are not limited to, a deep learning model, a neural network, and/or other machine learning model.

Likewise, using clinical data, a unimodal model is constructed and trained to extract clinical features that are predictive of the health condition. For example, a training dataset that includes clinical data of subjects that have the health condition and those that do not have the health condition can be used to train the unimodal model to extract clinical features that are predictive of the health condition. By way of example, clinical feature such as an abnormal heart function, low blood count levels, and/or other clinical features can be extracted as being predictive of disease A, based on learning from the training dataset. Examples of a unimodal model include but are not limited to, a deep learning model, a neural network, and/or other machine learning model.

Other types of modes of data can be used to construct other unimodal models. For example, while not shown, mode of data such as demographic data can be used to train a unimodal model to extract demographic features that could be predictive of the health condition.

A multi-modal model may be constructed using each of the unimodal models built using different data types or modes of data, and integrating the overall model (e.g., all unimodal model) predictions. In another aspect, a multi-modal model can be built by combining all features from all modes of data.

For each of the unimodal models developed using different modes of data separately as features, e.g., shown at 310, a ranking of important features can be constructed as shown at 312. Some models are inherently “transparent” and provide users with the most important features relevant for a model output. In other models such as deep neural networks, the display of such feature importance is not inherent to deep neural networks. In some embodiments, pipelines such as Local Interpretable Model-Agnostic Explanations (LIME) or SHapley Additive explanations (SHAP), but not limited to those techniques, can be used for ranking features. SHAP provides the Shapley regression values to rank the features relative to the tasks being learned and provides different techniques to compute very efficiently these values, especially in the presence of multi-collinearity among the features. LIME can generate global summary features from the local features that it has already generated from the given samples.

In some embodiments, ranking can be performed of all features of a multimodal model combining all features. For example, all features from all unimodal models can be combined, then ranked according to their importance. Importance here is measured by how much each of these features contributes to the overall prediction of the model.

312 shows a set of 5 features extracted from imaging data on top and 3 features extracted from demographic data on bottom using SHAP. Heatmap from left to right shows the feature importance regarding the outcome prediction, each dot represents the value of the feature for a single patient and whether the probability outcome was higher (left value for Shap explanation) or lower (right value for Shap explanation).

Each of the top features from each model can be used as a special phenotype or trait that has predictive association with the health condition, e.g., a disease. A stratification of given population can be implemented using the top feature where each patient or subject including the feature is part of the grouping associated with that feature. This process can be repeated for each of the top X features, where X is predefined.

For example, using the top image feature extracted or output by a unimodal trained on image data 304, a stratification is performed of a population where the stratification groups the subjects in the population having the top image feature into a subpopulation 314. Similarly, using the top genomic feature extracted or output by a unimodal trained on genomic data 306, a stratification is performed of a population where the stratification groups the subjects in the population having the top genomic feature into a subpopulation 316. Likewise, using the top clinical feature extracted or output by a unimodal trained on clinical data 3086, a stratification is performed of a population where the stratification groups the subjects in the population having the top clinical feature into a subpopulation 318.

A GWAS analysis is implemented on that specific subset of the population including the top X features. For example, the subpopulation 314 includes subjects that have any of top X image features, and a GWAS analysis can be performed on this subpopulation 314. This can be repeated for each of the X features in the different data modes. For example, the subpopulation 316 includes subjects that have any of top X genomic features, and a GWAS analysis can be performed on this subpopulation 316. Likewise, the subpopulation 318 includes subjects that have any of top X clinical features, and a GWAS analysis can be performed on this subpopulation 318.

Gene ontology enrichment analysis can be performed to discover SNPs focal associations with certain structural components given by the non-genetic data such as the image data and clinical data. Gene ontology enrichment (or any kind of gene set enrichment) helps bring SNPs to biological functions by asking whether the observed association is higher than would be expected given random sampling assessed by the hypergeometric test.

In some embodiments, the processing shown in FIGS. 3A-3B can be a closed loop process where the defined subpopulation is once again used to perform a stratification using the predictive features of a newly trained multimodal model using such subpopulation as shown at 320. For example, image data 304 of the subpopulation 314 is used to train a unimodal model of that mode at 310, to extract image features that are predictive of the health condition. For example, images of subjects in the subpopulation 314 that have the health condition and images of subjects in the subpopulation 314 that do not have the health condition can be labeled, and the labeled data can be used to train the unimodal model 310. The unimodal model outputs or extracts image features that are predictive of the health condition, which can be ranked 312. Ranked features can be used to perform stratification of a population into subpopulation 314.

Similarly, for example, genomic data 306 of the subpopulation 316 is used to train a unimodal model of that mode at 310, to extract genomic features that are predictive of the health condition. For example, genomics of subjects in the subpopulation 316 that have the health condition and genomics of subjects in the subpopulation 316 that do not have the health condition can be labeled, and the labeled data can be used to train the unimodal model 310. The unimodal model outputs or extracts genomic features that are predictive of the health condition, which can be ranked 312. Ranked features can be used to perform stratification of a population into subpopulation 316.

Likewise, for example, clinical data 308 of the subpopulation 318 is used to train a unimodal model of that mode at 310, to extract clinical features that are predictive of the health condition. For example, clinical data of subjects in the subpopulation 318 that have the health condition and clinical data of subjects in the subpopulation 318 that do not have the health condition can be labeled, and the labeled data can be used to train the unimodal model 310. The unimodal model outputs or extracts clinical features that are predictive of the health condition, which can be ranked 312. Ranked features can be used to perform stratification of a population into subpopulation 318.

In some embodiments, the loop can be repeated many times until not enough subjects are part of the subpopulation. For example, the loop 320 can be repeated until the number of subjects in the subpopulation (e.g., 314, 316, 318) falls below a threshold number. The threshold number can be predefined, for example, by a subject matter expert, for example, which can be based on feasibility of performing a GWAS.

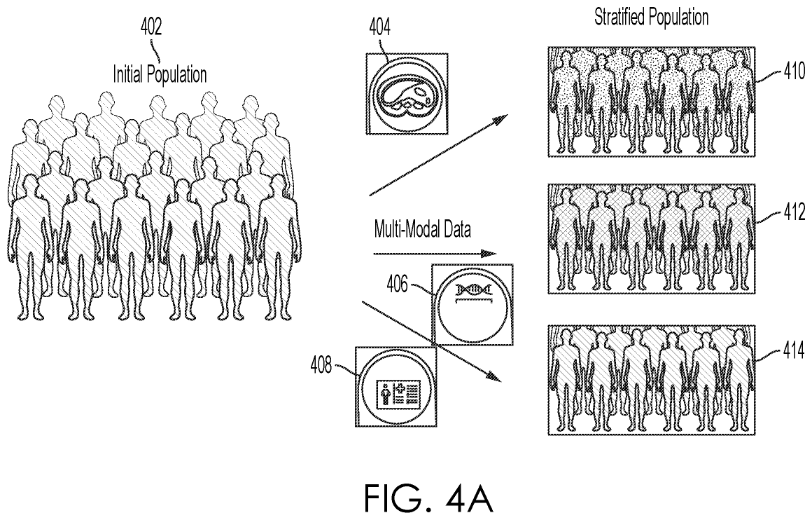

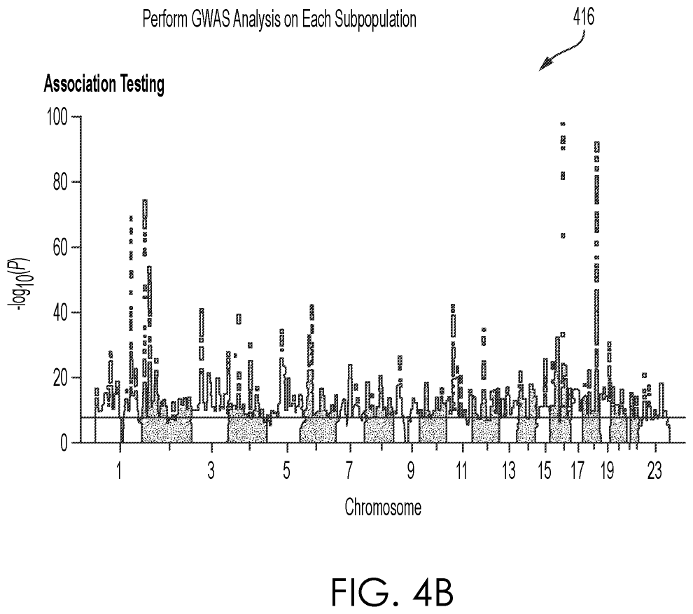

FIGS. 4A-4B illustrate an overview of GWAS on stratified population in some embodiments. As described above with reference to FIG. 2 and FIGS. 3A-3B, stratification can be performed on initial population 402 based on features extracted from multi-modal data 404, 406, 408 to form or define stratified population 410, 412, 414. On each of the stratified population 410, 412, 414, GWAS analysis can be performed, an example result of which is shown at 416 depicting a so called ‘Manhattan plot’ where SNPs enriched in the genome location are shown by a single point and a concentration of points forming a ‘skyscraper’ indicate an important position of genes associated with the trait.

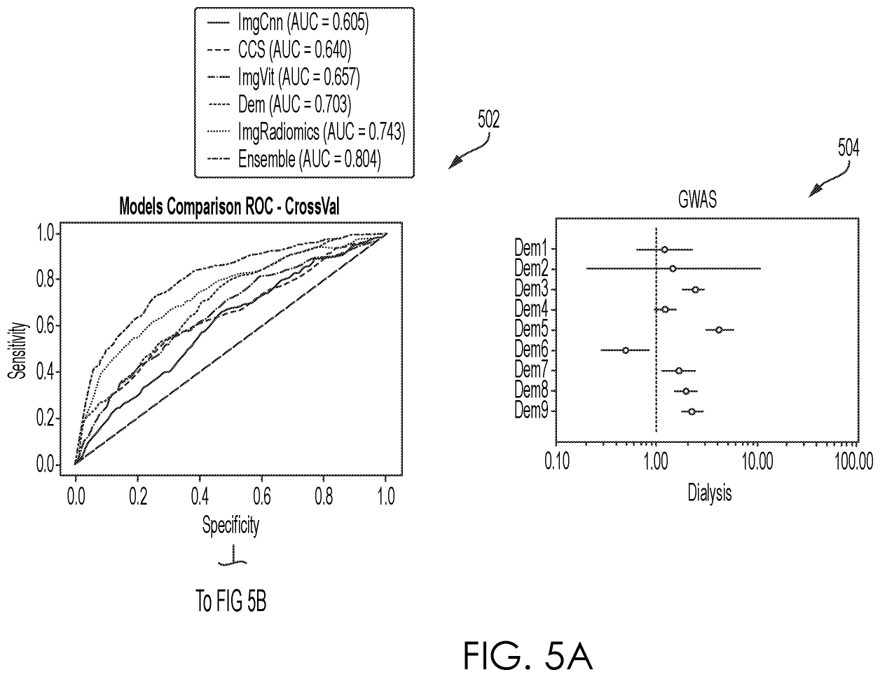

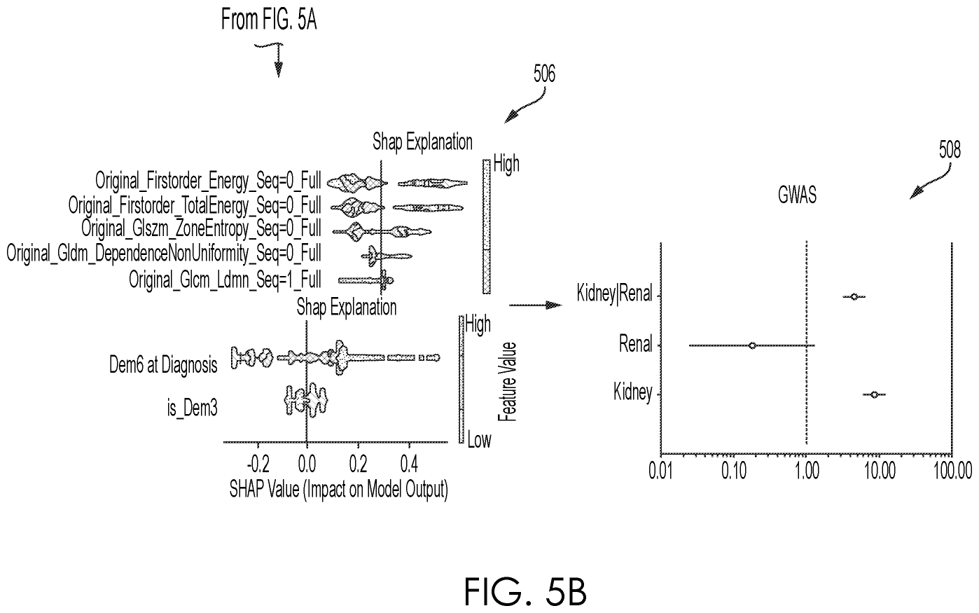

FIGS. 5A-5B illustrate a case study of an example population in some embodiments. Plot at 502 shows example results of model predictions (measured as AUC (Area Under the ROC (receiver operating characteristic) Curve) for each of the different data types and model pipelines. ‘CCS’ denotes clinical data, ‘Dem’ denotes demographic data, ‘Img’ imaging data and CNN, ViT and radiomics are different types of models ingesting imaging data. ‘Ensemble’ denotes the multimodal model. The chart at 504 shows an example Odds Ratio for each of the variables given the predicted trait in the overall population. The chart at 506 shows example SHAP values for imaging and demographic variables. The chart at 508 shows an example Odds Ratio for the Gene sets related to the described conditions after stratification of the population using the top feature in 506.

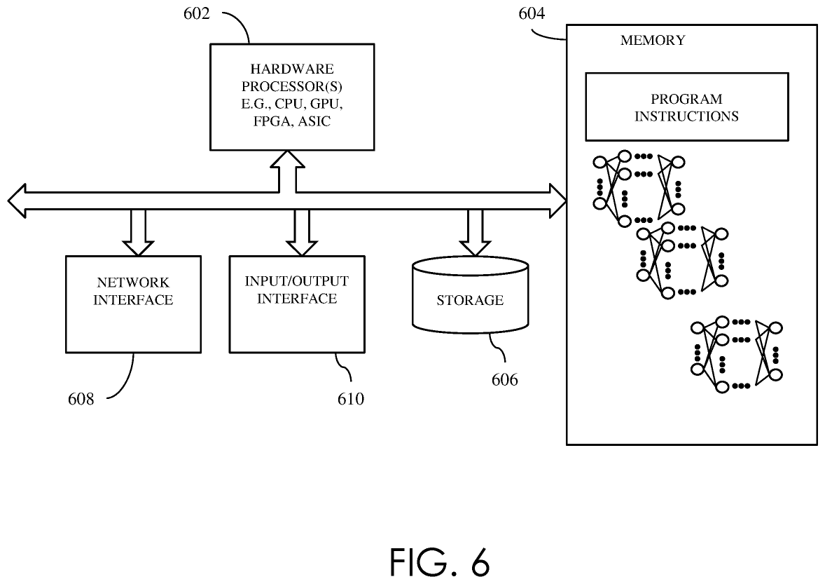

FIG. 6 is a diagram showing components of a system that can implement stratification of population using features of multimodal data in some embodiments. One or more hardware processors 602 such as a central processing unit (CPU), a graphic process unit (GPU), and/or a Field Programmable Gate Array (FPGA), an application specific integrated circuit (ASIC), and/or another processor, may be coupled with a memory device 604. A memory device 604 may include random access memory (RAM), read-only memory (ROM) or another memory device, and may store data and/or processor instructions for implementing various functionalities associated with the methods and/or systems described herein. One or more processors 602 may execute computer instructions stored in memory 604 or received from another computer device or medium. A memory device 604 may, for example, store instructions and/or data for functioning of one or more hardware processors 602, and may include an operating system and other program of instructions and/or data. One or more hardware processors 602 may construct a plurality of unimodal models, a unimodal model of the plurality of unimodal models trained based on a mode of data that is different from another mode of data used to train another one of the plurality of unimodal models, the plurality of unimodal models being trained to extract features that are predictive of a health condition. One or more hardware processors 602 may run the plurality of unimodal models, where sets of features are extracted, each set of the sets of features being associated with a mode of data. One or more hardware processors 602 may, for each set of the sets of features: rank the features in the set; perform a stratification of a population into a subpopulation according to the top ranked feature, where a top ranked feature is used as a special trait that has predictive association with the health condition; perform a genome-wide association study (GWAS) using genomic data associated with the subpopulation; and identify, based on the performed GWAS, at least one genomic variant associated with the health condition. Data used by one or more processors 602 may be stored in a storage device 606 or received via a network interface 608 from a remote device, and may be temporarily loaded into a memory device 604 for constructing unimodal models, performing stratification and/or performing GWAS analysis. The trained models may be stored on a memory device 604, for example, for running by one or more hardware processors 602. One or more hardware processors 602 may be coupled with interface devices such as a network interface 608 for communicating with remote systems, for example, via a network, and an input/output interface 610 for communicating with input and/or output devices such as a keyboard, mouse, display, and/or others.

The terminology used herein is for the purpose of describing particular embodiments only and is not intended to be limiting of the invention. As used herein, the singular forms “a”, “an” and “the” are intended to include the plural forms as well, unless the context clearly indicates otherwise. As used herein, the term “or” is an inclusive operator and can mean “and/or”, unless the context explicitly or clearly indicates otherwise. It will be further understood that the terms “comprise”, “comprises”, “comprising”, “include”, “includes”, “including”, and/or “having,” when used herein, can specify the presence of stated features, integers, steps, operations, elements, and/or components, but do not preclude the presence or addition of one or more other features, integers, steps, operations, elements, components, and/or groups thereof. As used herein, the phrase “in some embodiments” does not necessarily refer to the same embodiment, although it may. As used herein, the phrase “in one embodiment” does not necessarily refer to the same embodiment, although it may. As used herein, the phrase “in another embodiment” does not necessarily refer to a different embodiment, although it may. Further, embodiments and/or components of embodiments can be freely combined with each other unless they are mutually exclusive.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A computer-implemented method comprising:
constructing a plurality of unimodal models, a unimodal model of the plurality of unimodal models trained based on a mode of data that is different from another mode of data used to train another one of the plurality of unimodal models, the plurality of unimodal models being trained to extract features that are predictive of a health condition;
running the plurality of unimodal models, wherein sets of features are extracted, each set of the sets of features being associated with a mode of data; and
for each set of the sets of features:
ranking the features in the set;
performing a stratification of a population into a subpopulation according to a top ranked feature, wherein the top ranked feature is used as a special trait that has predictive association with the health condition;
performing a genome-wide association study (GWAS) using genomic data associated with the subpopulation; and
identifying, based on the performed GWAS, at least one genomic variant associated with the health condition.

2. The computer-implemented method of claim 1, further including:
retraining each of the plurality of unimodal models based on the respective subpopulation defined during the stratification; and
repeating the running, the ranking, the performing of the stratification, the performing of the GWAS, and the identifying steps.

3. The computer-implemented method of claim 2, further including iteratively performing the retraining and the repeating until the subpopulation meets a threshold population size.

4. The computer-implemented method of claim 1, wherein the performing of the stratification, the performing of the GWAS, and the identifying steps are performed for a threshold number of next top ranked features as the top ranked feature.

5. The computer-implemented method of claim 1, further including performing gene ontology enrichment analysis to discover biological functions associated to single nucleotide polymorphism (SNPs) using data associated with the subpopulation.

6. The computer-implemented method of claim 1, wherein multiple modes of data, each of which is used to train a respective different unimodal model in the plurality of unimodal models, include at least image data and clinical data.

7. The computer-implemented method of claim 1, wherein the health condition is a disease.

8. The computer-implemented method of claim 1, wherein the plurality of unimodal models are combined as a multimodal model, wherein the running the plurality of unimodal models includes running the multimodal model.

9. A computer program product comprising a computer readable storage medium having program instructions embodied therewith, the program instructions readable by a device to cause the device to:
construct a plurality of unimodal models, a unimodal model of the plurality of unimodal models trained based on a mode of data that is different from another mode of data used to train another one of the plurality of unimodal models, the plurality of unimodal models being trained to extract features that are predictive of a health condition;
run the plurality of unimodal models, wherein sets of features are extracted, each set of the sets of features being associated with a mode of data; and
for each set of the sets of features:
rank the features in the set;
perform a stratification of a population into a subpopulation according to a top ranked feature, wherein the top ranked feature is used as a special trait that has predictive association with the health condition;
perform a genome-wide association study (GWAS) using genomic data associated with the subpopulation; and
identify, based on the performed GWAS, at least one genomic variant associated with the health condition.

10. The computer program product of claim 9, wherein the device is further caused to:
retrain each of the plurality of unimodal models based on the respective subpopulation defined during the stratification; and
repeat running of the plurality of unimodal models, ranking of the features, performing of the stratification, performing of the GWAS study, and identifying of the at least one genomic variant associated with the health condition.

11. The computer program product of claim 10, wherein the device is further caused to iteratively perform retraining of the plurality of unimodal models and repeating of the running, ranking, performing of the stratification, until the subpopulation meets a threshold population size.

12. The computer program product of claim 9, wherein the device is further caused to perform the stratification, perform the GWAS, and identify at least one genomic variant, for a threshold number of next top ranked features as the top ranked feature.

13. The computer program product of claim 9, wherein the device is further caused to perform gene ontology enrichment analysis to discover biological functions associated to single nucleotide polymorphism (SNPs) using data associated with the subpopulation.

14. The computer program product of claim 9, wherein multiple modes of data, each of which is used to train a respective different unimodal model in the plurality of unimodal models, include at least image data and clinical data.

15. The computer program product of claim 9, wherein the health condition is a disease.

16. The computer program product of claim 9, wherein the plurality of unimodal models are combined as a multimodal model, wherein the device caused to run the plurality of unimodal models includes the device caused to run the multimodal model.

17. A system comprising:
at least one memory device; and
at least one computer processor configured to at least:
construct a plurality of unimodal models, a unimodal model of the plurality of unimodal models trained based on a mode of data that is different from another mode of data used to train another one of the plurality of unimodal models, the plurality of unimodal models being trained to extract features that are predictive of a health condition;
run the plurality of unimodal models, wherein sets of features are extracted, each set of the sets of features being associated with a mode of data; and
for each set of the sets of features:
rank the features in the set;
perform a stratification of a population into a subpopulation according to a top ranked feature, wherein the top ranked feature is used as a special trait that has predictive association with the health condition;
perform a genome-wide association study (GWAS) using genomic data associated with the subpopulation; and
identify, based on the performed GWAS, at least one genomic variant associated with the health condition.

18. The system of claim 17, wherein the at least one computer processor is further configured to:
retrain each of the plurality of unimodal models based on the respective subpopulation defined during the stratification; and
repeat running of the plurality of unimodal models, ranking of the features, performing of the stratification, performing of the GWAS study, and identifying of the at least one genomic variant associated with the health condition.

19. The system of claim 18, wherein the at least one computer processor is further configured to iteratively perform retraining of the plurality of unimodal models and repeating of the running, the ranking, the performing of the stratification, until the subpopulation meets a threshold population size.

20. The system of claim 19, wherein the at least one computer processor is further configured to perform the stratification, perform the GWAS, and identify at least one genomic variant, for a threshold number of next top ranked features as the top ranked feature.
